(19) United States
(12) Patent Application Publication
(10) Pub. No.: US 2011/0263720 A1
(43) Pub. Date: Oct. 27, 2011

(54) SYNTHESIS OF ALPHA-TOCOPHEROLQUINONE DERIVATIVES, AND METHODS OF USING THE SAME

(75) Inventors: Steven D. PAISLEY, Danbury, CT (US); Jason M. LePree, Demarest, NJ (US); Amale Hawi, Ridgefield, CT (US); Michael Eckhoff, Ridgefield, CT (US)

(73) Assignee: Penwest Pharmaceuticals Co., Patterson, NY (US)

(21) Appl. No.: 13/044,056

(22) Filed: Mar. 9, 2011

Related U.S. Application Data
(60) Provisional application No. 61/312,185, filed on Mar. 9, 2010.

(51) Int. Cl. A61K 31/122; C07C 49/497; A61P 25/00; A61P 3/10; A61P 25/28; A61P 25/18; A61P 35/00; C07C 45/60; A61P 25/08

(52) U.S. Cl. 514/678; 568/323; 568/308

(57) ABSTRACT

The present invention is directed to a method of synthesizing a compound of Formula I:

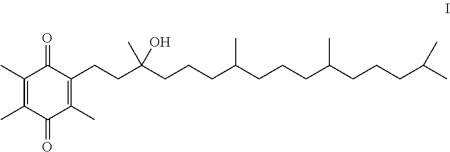

the method comprising oxidizing alpha-tocopherol with a metal salt oxidizing agent to form the compound of Formula I, wherein the stoichiometric ratio (mol/mol) of metal salt oxidizing agent/alpha-tocopherol is 1.6 to 4. The invention is also directed to a method of synthesizing a compound of Formula I, the method comprising (a) hydrolyzing alpha-tocopheryl acetate in the presence of a base; (b) neutralizing the hydrolyzing of (a), thereby forming alpha-tocopherol; and (c) oxidizing the alpha-tocopherol of (b) with a metal salt oxidizing agent to form the compound of Formula I, wherein the stoichiometric ratio (mol/mol) of metal salt oxidizing agent/alpha-tocopherol is 1.6 to 4.

### CROSS REFERENCE TO RELATED APPLICATION

This application claims priority to U.S. Provisional Application No. 61/312,185, that was filed on Mar. 9, 2010. The content of U.S. Provisional Application No. 61/312,185 is incorporated by reference herein in its entirety.

### FIELD OF THE INVENTION

The present invention relates to a method of synthesizing a compound of Formula I:

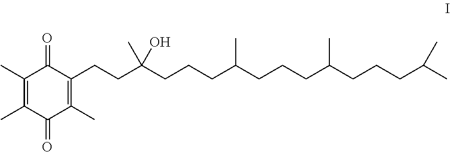

the method comprising oxidizing alpha-tocopherol with a metal salt oxidizing agent to form the compound of Formula I, wherein the stoichiometric ratio (mol/mol) of the metal salt oxidizing agent/alpha-tocopherol is 1.6 to 4. The invention is also directed to a method of synthesizing a compound of Formula I, the method comprising (a) hydrolyzing alpha-tocopheryl acetate in the presence of a base; (b) neutralizing the hydrolyzing of (a), thereby forming alpha-tocopherol; and (c) oxidizing the alpha-tocopherol of (b) with a metal salt oxidizing agent to form the compound of Formula I, wherein the stoichiometric ratio (mol/mol) of the metal salt oxidizing agent/alpha-tocopherol is 1.6 to 4.

### BACKGROUND OF THE INVENTION

### Background Art

R,R,R-alpha-tocopherolquinone is under development for treatment of symptoms associated with mitochondrial diseases. It occurs naturally in man but in very low concentrations (<0.2 μg/mL). It is similar to Coenzyme Q (CoQ¹⁰, ubiquinone), a lipid-soluble component of cell membranes that functions as both a coenzyme in the mitochondrial electron transport chain and, in its reduced form, as an antioxidant. Lenaz, G., et al., Mitochondrion 7S:S8-33 (2007). In vitro experiments with R,R,R-alpha-tocopherolquinone have shown it to be far more potent than CoQ¹⁰ in enhancing mitochondrial function. Several in vitro and in vivo studies have demonstrated the potential benefit of CoQ¹⁰ in a variety of diseases of the nervous system (Parkinson Disease and Huntington Disease) and mitochondrial diseases (mitochondrial encephalopathy, lactic acidosis and stroke-like episodes (MELAS), Friedreich Ataxia, Leber hereditary optic neuropathy (LHON), Kearns-Sayre syndrome, and CoQ¹⁰ deficiency syndromes). See, e.g., Shults, C. W., et al., Arch. Neurol. 59:1541-50 (2002); Beal, M. F., et al., Biofactors 9:261-6 (1999); Haas, R. H., Mitochondrion 7S:5136-45 (2007); and Cooper, J. M., et al., Mitochondrion 7S:S127-35 (2007).

Idebenone is a similar CoQ¹⁰ analog that has demonstrated safety and potential utility for conditions including Alzheimer's Disease, Friedreich Ataxia, Huntington's Disease and MELAS. Gutzmann, H., et al., J. Neural Transm Suppl. 54:301-10 (1998); Di Prospero, N. A., et al., Lancet Neurol. 6:878-86 (2007); Ranen, N. G., et al., Mov. Disord. 11:549-54 (1996).

Alpha-tocopherolquinone can be synthesized by oxidation of alpha-tocopherol. Oxidation of alpha-tocopherol with ferric chloride has been reported since 1937 (John, Z., Physiol. Chem., 250 (1937)). Emmerie and Engels described a procedure to use oxidation with ferric chloride as a quantitative measurement for the amount of alpha-tocopherol in materials (Emmerie and Engel, Rec. Tray. Chim., 57:135 (1938)). Skinner has described some side-products that can occur during oxidation of alpha-tocopherol (Skinner, W. A., Ph.D. Thesis, University of Texas at Austin (1952)).

However, traditional methods of synthesizing alpha-tocopherolquinone often result in higher than desired concentrations of side-products. For example, commercial R,R,R-alpha-tocopheryl acetate is invariably contaminated with varying amounts of beta-tocopheryl acetate and gamma-tocopheryl acetate. A consequence of these impurities is that oxidation of alpha-tocopherol to form alpha-tocopherolquinone also results in oxidation of these impurities and formation of beta-tocopherolquinone and gamma-tocopherolquinone. Thus, new methods of synthesizing alpha-tocopherolquinone are needed to reduce the level of side-products such as beta-tocopherolquinone and gamma-tocopherolquinone.

### BRIEF SUMMARY OF THE INVENTION

The invention is directed to a method of synthesizing a compound of Formula I:

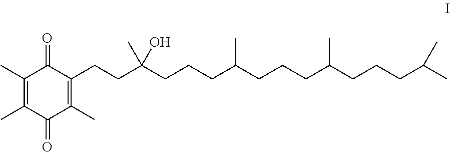

or a stereoisomer thereof, the method comprising oxidizing alpha-tocopherol with a metal salt oxidizing agent to form the compound of Formula I, wherein the stoichiometric ratio (mol/mol) of metal salt oxidizing agent/alpha-tocopherol is 1.6 to 4.

The present invention is also directed to a method of synthesizing a compound of Formula I:

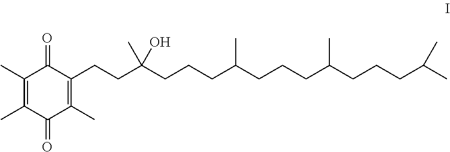

or a stereoisomer thereof, the method comprising: (a) hydrolyzing alpha-tocopheryl acetate in the presence of a base; (b) neutralizing the hydrolyzing of (a), thereby forming alpha-tocopherol; and (c) oxidizing the alpha-tocopherol of (b) with a metal salt oxidizing agent to form the compound of Formula I, wherein the stoichiometric ratio (mol/mol) of metal salt oxidizing agent/alpha-tocopherol is 1.6 to 4.

In some embodiments, the metal salt oxidizing agent is an iron halide such as, e.g., FeCl₃. In some embodiments, the metal salt oxidizing agent is serially added in more than one portion. In some embodiments, the method further comprises washing the product of (c) with an aqueous solution.

In some embodiments, the stoichiometric ratio (mol/mol) of metal salt oxidizing agent/alpha-tocopherol is 2.5 to 3.5. In some embodiments, the metal salt oxidizing agent is added in an amount sufficient to oxidize 70% (mol/mol) to 98% (mol/mol) of the alpha-tocopherol to the compound of Formula I.

In some embodiments, the base is potassium hydroxide or sodium hydroxide. In some embodiments, the neutralizing is by addition of an acid.

In some embodiments, the alpha-tocopheryl is dissolved in an alcohol before hydrolysis. In some embodiments, the hydrolysis occurs at about 5° C. to about 20° C.

In some embodiments, alpha-tocopheryl acetate is R,R,R-alpha-tocopheryl acetate. In some embodiments, the invention is directed to a compound of Formula I, made by one of the methods of syntheses described herein. In some embodiments, the compound of Formula I is:

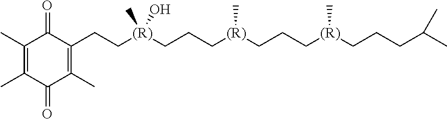

i.e., R,R,R-alpha-tocopherolquinone, or a stereoisomer thereof.

In some embodiments, the method of the present invention produces a composition having less than 2% (mol/mol) gamma-tocopherolquinone relative to the compound of Formula I. In some embodiments, the composition has less than 0.7% (mol/mol) gamma-tocopherolquinone, or less than 0.2% (mol/mol) gamma-tocopherolquinone relative to the compound of Formula I. In some embodiments, the compound of Formula I is greater than 70% (wt/wt) of the composition. In some embodiments, the composition has less than 20% (wt/wt) of alpha-tocopheryl acetate, alpha-tocopherol, beta-tocopherol, beta-tocopherolquinone, gamma-tocopherol, or combinations thereof.

In some embodiments, the method of the present invention further comprising adding a pharmaceutically acceptable excipient. In some embodiments, the invention is directed to an oral dosage form comprising the compound of Formula I, made by the method of the present invention.

The present invention is also directed to a method of treating a mitochondrial disorder, modulating one or more energy biomarkers, normalizing one or more energy biomarkers, or enhancing one or more energy biomarkers, the method comprising administering to a subject a therapeutically effective amount or effective amount of a composition made by the methods of the present invention, the composition comprising a compound of Formula I:

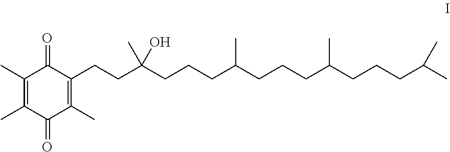

or a stereoisomer thereof, wherein the composition has less than 2% (mol/mol) gamma-tocopherolquinone relative to the compound of Formula I. In some embodiments, the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Leigh Disease; Kearns-Sayre Syndrome (KSS); Friedreich's Ataxia (FA); other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; other neurological diseases; epilepsy; genetic diseases; Huntington's Disease; mood disorders; schizophrenia; bipolar disorder; age-associated diseases; macular degeneration; diabetes; and cancer. In some embodiments, the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Leigh Disease; Kearns-Sayre Syndrome (KSS); and Friedreich's Ataxia (FA).

In some embodiments, the energy biomarker is selected from the group consisting of: lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H) levels; NADPH (NADPH+H) levels; NAD levels; NADP levels; ATP levels; reduced coenzyme Q (CoQ^{red}) levels; oxidized coenzyme Q (CoQ^{OX}) levels; total coenzyme Q (CoQ^{tot}) levels; oxidized cytochrome C levels; reduced cytochrome C levels; oxidized cytochrome C/reduced cytochrome C ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; levels of oxygen consumption (VO₂); levels of carbon dioxide output (VCO₂); respiratory quotient (VCO₂/VO₂); exercise tolerance; and anaerobic threshold.

In some embodiments, the subject is selected from the group consisting of: a subject with a mitochondrial disease; a subject undergoing strenuous or prolonged physical activity; a subject with chronic energy problems; a subject with chronic respiratory problems; a pregnant female; a pregnant female in labor; a neonate; a premature neonate; a subject exposed to an extreme environment; a subject exposed to a hot environment; a subject exposed to a cold environment; a subject exposed to an environment with lower-than-average oxygen content; a subject exposed to an environment with higher-than-average carbon dioxide content; a subject exposed to an environment with higher-than-average level of air pollution; a subject with lung disease; a subject with lower-than-average lung capacity; a tubercular patient; a lung cancer patient; an emphysema patient; a cystic fibrosis patient; a subject recovering from surgery; a subject recovering from illness; a subject undergoing acute trauma; a subject in shock; a subject requiring acute oxygen administration; a subject requiring chronic oxygen administration; an elderly subject; an elderly subject experiencing decreased energy; and a subject suffering from chronic fatigue.

### BRIEF DESCRIPTION OF THE FIGURE

FIG. 1 summarizes a method of converting alpha-tocopherol acetate to alpha-tocopherolquinone using potassium hydroxide, and TBME.

### DETAILED DESCRIPTION OF THE INVENTION

The present invention is directed to a method for limiting the formation of beta- and gamma-tocopherolquinone through control of the stoichiometry of the metal salt oxidizing agent. The inventors have found that the rate of reaction of alpha-tocopherol with ferric chloride is faster than the rate of reaction of either beta- or gamma-tocopherol. Thus, if the oxidation process uses a limited amount of metal salt oxidizing agent, such that some of the tocopherols are not oxidized before the metal salt oxidizing agent is exhausted, preferential oxidation of the alpha-isomer can be achieved.

### Synthesis

The present invention is directed to a method of synthesizing compound of Formula I:

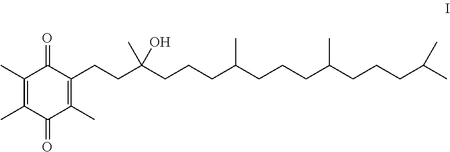

or a stereoisomer thereof, the method forming a reduced amount of gamma-tocopherolquinone. In some embodiments, the method comprises oxidizing alpha-tocopherol with a metal salt oxidizing agent to form the compound of Formula I, wherein the stoichiometric ratio (mol/mol) of metal salt oxidizing agent/alpha-tocopherol is 1.6 to 4. In some embodiments, the method comprises (a) hydrolyzing alpha-tocopheryl acetate in the presence of a base; (b) neutralizing the hydrolyzing of (a), thereby forming alpha-tocopherol; and (c) oxidizing the alpha-tocopherol of (b) with a metal salt oxidizing agent to form the compound of Formula I, wherein the stoichiometric ratio (mol/mol) of metal salt oxidizing agent/alpha-tocopherol is 1.6 to 4.

The alpha-tocopheryl acetate starting material employed in the present invention can be isolated from various organisms, or can be chemically synthesized. In some embodiments, alpha-tocopherol is isolated from a plant, e.g., vegetable oils such as palm oil, sunflower, corn, soybean, and olive oil, nuts, seabuckthorn berries, kiwifruit, wheat germ, whole grains, peanut butter, or green leafy vegetables, and is then chemically converted to acetate. In some embodiments, the alpha-tocopheryl acetate starting material is in a relatively impure form, i.e., it is contaminated with beta-tocopheryl acetate and/or gamma-tocopheryl acetate. For example, in some embodiments, the alpha-tocopheryl acetate contains greater than 1%, 2%, 3%, 5%, 8%, 10%, or 15% beta-tocopheryl acetate, gamma-tocopheryl acetate, or a combination thereof. Thus, the method of the present invention can be of particular benefit when an impure form of alpha-tocopheryl acetetate is used as a starting material, since it will reduce the formation of beta-tocopherolquinone and/or gamma-tocopherolquinone that would otherwise occur in the oxidation reaction. Thus, the method of the present invention allows the use of relatively impure forms of alpha-tocopheryl acetate, without the need of further chromotographic methods to remove the beta-tocopherolquinone and/or gamma-tocopherolquinone.

The intermediates that result from the hydrolysis of alpha-tocopheryl acetate and/or the neutralization of hydrolysis can be isolated prior to the oxidation of the alpha-tocopherol, or the oxidation can be performed without isolating the alpha-tocopherol. Hydrolysis of alpha-tocopheryl acetate to alpha-tocopherol can be accomplished by charging the appropriate quantity of alpha-tocopheryl acetate into a reaction vessel, and then adding a base. The term “hydrolyzing alpha-tocopheryl acetate” involves the conversion, i.e., hydrolysis, of alpha-tocopheryl acetate to form alpha-tocopherol.

Hydrolysis reactions to remove acetate groups are known in the art, and include hydrolysis by addition of a base. In some embodiments, the base hydrolysis is accomplished by, but not limited to, addition of a strong base. Strong bases are known to those in the art, and can include, but are not limited to, alkali and earth alkali metal hydroxides and alkoxides such as sodium hydroxide, potassium hydroxide, lithium hydroxide, rubidium hydroxide, calcium hydroxide, strontium hydroxide, barium hydroxide sodium alkoxide, potassium alkoxide, lithium alkoxide, or rubidium alkoxide, calcium alkoxide, strontium alkoxide, or barium alkoxide. As used herein, the term “alkoxide” refers to lower alkoxides, e.g., methoxide, ethoxide, propoxide, etc. Thus, in some embodiments of the present invention, the hydrolysis occurs via addition of, e.g., sodium hydroxide, potassium hydroxide, sodium methoxide, potassium methoxide, sodium ethoxide and/or potassium ethoxide. Additional strong bases can include, but are not limited to, Group 1 salts of carbanions, amides, and hydrides such as butyl lithium (n-BuLi), lithium diisopropylamide (LDA) (C₆H₁₄LiN), lithium diethylamide (LDEA), sodium amide (NaNH₂), sodium hydride (NaH), and lithium bis(trimethylsilyl)amide.

In some embodiments, the hydrolysis reaction is performed in the presence of additional solvents, e.g., an alcohol such as methanol, ethanol, or propanol. In some embodiments, the hydrolysis reaction is performed at a reduced temperature, e.g., from about −20° C. to about 35° C., or from about −10° C. to about 30° C., or from about 0° C. to about 20° C., or from about 5° C. to about 15° C. In some embodiments, the alpha-tocopheryl acetate is cooled prior to the hydrolysis, and is maintained at the cooled temperature throughout the hydrolysis reaction. In some embodiments, the hydrolysis reaction is agitated, such as, e.g., by mechanical means, throughout the reaction period.

The hydrolysis reaction can be allowed to proceed until the alpha-tocopheryl acetate is almost completely hydrolyzed. The reaction can be checked for completeness by sampling and analyzing the batch by means known to those in the art, e.g., by HPLC. In some embodiments, the reaction is complete when alpha-tocopheryl acetate is less than 2%, less than 1.5%, less than 1%, or less than 0.5% area percent at 205 nm as measured by HPLC.

In some embodiments, the hydrolysis reaction can be neutralized, i.e., quenched, to stop the hydrolysis reaction. In some embodiments, the method of the present invention can involve the neutralization of the hydrolysis reaction by addition of an acid. As used herein, the term “acid” refers to any compound or composition capable of lowering the pH of the hydrolysis reaction to less than a pH of 7. For example, in some embodiments the acid is a stong acid such as, but not limited to, hydrogen halides and their solutions, such as hydrochloric acid (HCl) and hydrobromic acid (HBr), sulfuric acid (H₂SO₄), nitric acid (HNO₃), phosphoric acid (H₃PO₄), chromic acid (H₂CrO₄), acetic acid, citric acid, Lonnie acid, gluconic acid, lactic acid, oxalic acid, or tartaric acid. The acids can be added to the reaction vessel in an amount sufficient to reduce the pH of the hydrolysis reaction to below a pH of 7, 6 or 5.

A metal salt oxidizing agent is added to the product of the hydrolysis reaction to facilitate the oxidation of the alpha-tocopherol to a compound of Formula I. Metal salt oxidizing agents are known to those in the art, and can include, but are not limited to transition metals with halides, e.g., chromium halides, manganese halides, iron halides, copper halides, paladium halides, silver halides, cadmium halides, and the like; transition metal oxides, e.g., silver oxide; permanganate ions; ferricyanide ions; nitric acid; iodine; bromine; hypochlorite; peroxides, and oxygen with a free radical initiator. In some embodiments, an electrochemical cell can be used in place of a metal salt oxidizing agent to oxidize alpha-tocopherol. One of skill in the art can calculate the time and energy required for an electrochemical cell to achieve an amount of oxidation comparable to the methods of synthesis described herein. The term “halides” refer to a halogen atom ion bearing a negative charge, the halide anions selected from the group consisting of fluoride (F⁻), chloride (Cl⁻), bromide (Br⁻), and iodide (I⁻).

Thus, the term metal salt oxidizing agent can include, e.g., FeCl₃. In some embodiments, the metal salt oxidizing agent is dissolved in a solvent solution, e.g., the metal salt oxidizing agent is in an aqueous solution, e.g., water or a water/alcohol solution.

The metal salt oxidizing agent can be added to alpha-tocopherol solution in various ways. For example, the metal salt oxidizing agent can be added to the quenched hydrolysis reaction at a single point in time, in a single portion, and/or stirred by mechanical mixing for the duration of the oxidation reaction. In some embodiments, the metal salt oxidizing agent can be added to the product of the hydrolysis reaction slowly over a prolonged period of time, e.g., over 1, 2, 10, 15, 20, or 30 minutes. Alternatively, the metal salt oxidizing agent can be added in multiple portions, i.e., serial additions or aliquots, wherein the metal salt oxidizing agent is added and the mixture is allowed to settle. In some embodiments, the aqueous layer is removed prior to addition of the next portion of metal salt oxidizing agent. In some embodiments, four portions are added, with the aqueous layer being removed before addition of any subsequent portions of metal salt oxidizing agent.

In some embodiments, the alpha-tocopherolquinone solution resulting from the addition of the metal salt oxidizing agent can be washed one or more times with an aqueous wash, e.g., water, buffer (e.g., sodium bicarbonate), or other aqueous solution (e.g., an aqueous salt solution, i.e., sodium or potassium chloride), to remove impurities and the metal salt oxidizing agent. For example, an aqueous wash can be performed at the termination of the oxidation reaction. In some embodiments, one or more washes are used, e.g., 2, 3, 4, 5 or more washes are used. The wash can then be discarded, or alternatively, can be recycled and/or reused. In some embodiments, an aqueous wash is performed prior to addition of one or more portions of metal salt oxidizing agent, and the aqueous layer is removed to waste between each portion.

Unless designated otherwise, the term “stoichiometric ratio” refers to the total ratio of the moles of metal salt oxidizing agent relative to the moles of alpha-tocopherol in the oxidation reaction. Thus, for a stoichiometric ratio in an oxidation reaction which includes serial additions of multiple portions of metal salt oxidizing agent, the term stoichiometric ratio would refer to the total additive amount of moles of metal salt oxidizing agent in all the multiple portions added to the oxidation reaction. In some embodiments the stoichiometric ratio is 1.6 to 4, 1.8 to 4, 2 to 4, 2.2 to 4, 2.5 to 4, 3 to 4, 3.5 to 4, or 3.8 to 4. In some embodiments, the stoichiometric ratio of metal salt oxidizing agent/alpha-tocopherol is 1.7 to 4, 1.8 to 3, or 2 to 2.5. In some embodiments the stoichiometric ratio of metal salt oxidizing agent/alpha-tocopherol is 2 to 4.5, 2.5 to 4, or 2.7 to 3.2.

In some embodiments, the oxidation reaction contains serial additions of multiple portions of metal salt oxidizing agent, wherein the individual portion of metal salt oxidizing agent contains a stoichiometric ratio of metal salt oxidizing agent/alpha-tocopherol of greater than 0.2, greater than 0.4, greater than 0.6, greater than 0.8, greater than 1, greater than 1.2, greater than 1.5, greater than 2, greater than 2.5, greater than 3, greater than 3.5, or greater than 4. In some embodiments, the stoichiometric ratio of metal salt oxidizing agent/alpha-tocopherol in each individual portion is 0.2 to 4.0, 0.5 to 3.5, 1.0 to 3.0 or 1.5 to 2.5. In some embodiments, the stoichiometric ratio of metal salt oxidizing agent/alpha-tocopherol in each individual portion is 0.2, 0.4. 0.5, 0.6, 0.7, 0.75, 0.8, 0.9, 1, 1.2, 1.4, 1.6, 2, 2.5, or 3.0. For example, in some embodiments, three or four portions of metal salt oxidizing agent are used, each portion having a stoichiometric ratio of metal salt oxidizing agent/alpha-tocopherol in each individual portion of 0.5, 0.6, 0.7, 0.75, 0.8, or 0.9. In some embodiments, multiple portions of metal salt oxidizing agent are used, each subsequent portion having a decreased stoichiometric ratio of metal salt oxidizing agent/alpha-tocopherol, e.g., the first portion has a ratio of 0.8, the second portion has a ratio of 0.7, and the third portion has a ratio of 0.6. Alternatively, multiple portions of metal salt oxidizing agent can be used with each subsequent portion having a increased stoichiometric ratio of metal salt oxidizing agent/alpha-tocopherol, e.g., the first portion has a ratio of 0.6, the second portion has a ratio of 0.7, and the third portion has a ratio of 0.8.

In some embodiments, the metal salt oxidizing agent is added in an amount sufficient to oxidize 70% (mol/mol) to 98% (mol/mol) of the alpha-tocopherol to the compound of Formula I. In some embodiments, the metal salt oxidizing agent is added in an amount sufficient to oxidize 75%, 85%, 87%, 90%, 92%, 95% or 97% (mol/mol) to the compound of Formula I.

In some embodiments, more than one solvent can be added to the product of the hydrolysis reaction. Such solvents can include, e.g., tert-butyl methyl ether (TBME), diethyl ether, or other solvents immisicble with water.

Oxidation of alpha-tocopherol can be carried out by adding a metal salt, e.g., ferric chloride, to the alpha-tocopherol. The alpha-tocopherol can be dissolved in methanol, ethanol, or acetone, with the metal salt, with or without water. In some embodiments, the reaction proceeds in a homogeneous solution.

In other embodiments, oxidation of alpha-tocopherol can be carried out by dissolving alpha-tocopherol in an ether, e.g., tert-butyl methyl ether (TBME), and dissolving the metal salt in water or a mixture of water and ethanol, preferably 2:1 of water:ethanol. The alpha-tocopherol and the metal salt are then mixed, the resulting mixture of metal salt/alpha-tocopherol consisting of two phases: (i) an organic phase consisting predominantly of alpha-tocopherol, and/or alpha-tocopherolquinone dissolved in TBME, and (ii) a predominantly aqueous phase containing ferric and ferrous chlorides, thus forming a two phase reaction system.

In the two phase reaction system, the metal salt can be serially added in multiple portions, wherein, a first portion of metal salt is added, followed by removal of the first portion and addition of a second portion of metal salt. Using serial portion addition, the metal salt of the first portion oxidizes the alpha-tocopherol and the spent oxidizer enters the aqueous phase and is predominantly removed to waste before addition of the subsequent portion.

The oxidation reaction can be allowed to proceed until the alpha-tocopherol is almost completely oxidized. The reaction can be checked for completeness by sampling and analyzing the batch by means known to those in the art, e.g., by HPLC. In some embodiments, the reaction is complete when alpha-tocopherol is less than about 60%, 55%, 50%, 45%, 40%, 35%, 30%, 25%, 20%, 15%, 10%, 5%, 4%, 3%, 2%, 1%, 0.5% or 0.2% area percent at 205 nm as measured by HPLC. In some embodiments, the sample is analyzed by HPLC using a C6-Phenyl column (Phenomenex, Torrance, Calif.) eluted with a mixture of acetonitrile and water.

Alternatively, the oxidation reaction can be allowed to proceed, while measuring the formation of one or more of the undesired side-products are formed. It has been found that there exists selective oxidation during the oxidation of alpha-tocopherol to alpha-tocopherolquinone. For example, one or more side-products is produced at a relatively reduced rate relative to the production of alpha-tocopherolquinone at the beginning of the oxidation reaction. Thus, in some embodiments, the oxidation reaction can be allowed to proceed until the level of one or more side-products reaches a predetermined level, e.g., 10%, 8%, 6%, 5%, 4%, 3%, 2%, or 1% area percent at 261 nm as measured by HPLC. In some embodiments, no single side product is greater than 5%, 4%, 3%, 2%, 1%, or 0.5% area percent at 261 nm as measured by HPLC. These undesired side-products can include, but are not limited to, beta-tocopherolquinone, gamma-tocopherolquinone, delta-tocopherolquinone, and combinations thereof. In some embodiments, the side-product is gamma-tocopherolquinone. In some embodiments, the reaction is considered to be complete when one or more of the side-products is less than about 10%, 8%, 6%, 5%, 4%, 3%, 2%, or 1% area percent at 261 nm as measured by HPLC.

Various means can be used to purify the alpha-tocopherolquinone from any solvents which can be present. Solvent extraction methods are known in the art. For example, in some embodiments, selective evaporation can be used to evaporate the solvent. In some embodiments, a rotary evaporator can be used to remove the solvent under vacuum. In some embodiments, the compound of Formula I is placed in an organic solvent such as n-heptane for storage and/or further processing.

In some embodiments, a metal chelating agent is used. Examples of metal chelating agents are known to those in the art and can include acrylic polymers, ascorbic acid, tetrasodium iminodisuccinate, citric acid, dicarboxymethylglutamic acid, ethylenediaminedisuccinic acid (EDDS), ethylenediaminetetraacetic acid (EDTA), methylene phosphonic acid), malic acid, or nitrilotriacetic acid (NTA). Additional means can be used to separate various undesired side-products, as well as the reaction material, the techniques including, but not limited to silica columns.

### Compositions

In some embodiments, the invention is directed to a method of synthesizing a compound of Formula I:

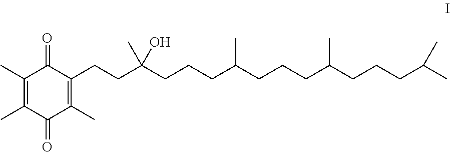

or a stereoisomer thereof, wherein the synthesis results in a composition having less than 2% (mol/mol) gamma-tocopherolquinone relative to the compound of Formula I.

The invention also includes all stereoisomers of the compounds, including diastereomers and enantiomers. The invention also includes mixtures of stereoisomers in any ratio, including, but not limited to, racemic mixtures. Unless stereochemistry is explicitly indicated in a structure, the structure is intended to embrace all possible stereoisomers of the compound depicted. If stereochemistry is explicitly indicated for one portion or portions of a molecule, but not for another portion or portions of a molecule, the structure is intended to embrace all possible stereoisomers for the portion or portions where stereochemistry is not explicitly indicated.

There are three stereocenters in alpha-tocopherol and/or alpha-tocopherolquinone, resulting in eight stereoisomers: R,R,R; S,R,R; R,S,R; R,R,S; R,S,S; S,R,S; S,S,R; and/or S,S,S. Each of these stereoisomers, either individually, or in any combination of racemic mixture, is included in the present invention. Thus, the compound of Formula I can also include any specific stereoisomer. In some embodiments, the compound of the present invention can be in the R,R,R conformation, as described by the formula:

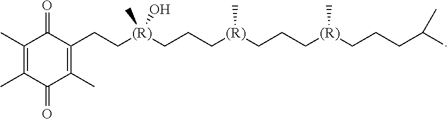

In some embodiments, a single stereoisomer is present, e.g., R,R,R, in the composition of the present invention. In some embodiments, at least 70%, 80%, 90%, 95%, or 99% (mol/mol) of the compounds of Formula I have the same stereochemistry, e.g., R,R,R. In some embodiments, the compounds of Formula I are in a racemic mixture, containing two or more different stereocenters.

The present invention can be directed to a method of synthesizing compositions having low amounts of gamma-tocopherolquinone. Gamma-tocopherolquinone can be represented by the formula:

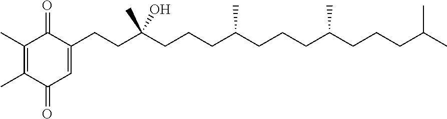

Compositions described herein car contain various amounts of gamma-tocopherolquinone. In some embodiments, the composition has less than 2%, less than 1.5%, less than 1.0%, less than 0.8%, less than 0.6%, less than 0.4%, or less than 0.2% (mol/mol) of gamma-tocopherolquinone relative to the compound of Formula I. In some embodiments, the composition has less than 0.2% (mol/mol) gamma-tocopherolquinone relative to the compound of Formula I. In some embodiments, the composition has less than 0.18%, less than 0.16%, less than 0.14%, or less than 0.10% (mol/mol) gamma-tocopherolquinone relative to the compound of Formula I. In some embodiments, gamma-tocopherolquinone is not detected in the composition using techniques currently known in the art.

### Methods of Using

In some embodiments, the compound of Formula I can be used to treat one or more conditions in a subject. The composition can comprise other materials and compounds in addition to the compound of Formula I. For example, the composition can comprise additional tocopheryl derivatives. In some embodiments, the tocopheryl derivatives can be less than 50% (wt/wt) of the total composition. In some embodiments, the composition has less than 20% (wt/wt) of alpha-tocopheryl acetate, alpha-tocopherol, beta-tocopherol, beta-tocopherylquinone, gamma-tocopherolquinone, or combinations thereof. In some embodiments, the composition has less than 15% (wt/wt), less than 10% (wt/wt), or 1% to 10% (wt/wt) alpha-tocopheryl acetate, alpha-tocopherol, beta-tocopherol, beta-tocopherylquinone, gamma-tocopherolquinone, or combinations thereof.

One of skill in the art can recognize that various excipients, flavorants, colorants, and/or rate-releasing agents can be added to the composition. In some embodiments, the composition is a pharmaceutically acceptable composition. In some embodiments, the composition further comprises a pharmaceutically acceptable excipients. As used herein, “excipient” refers to a substance, or mixture of substances, that is used in the formulation of compositions of the present invention, to give desirable physical characteristics to the formulation. As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. In some embodiments, the term “pharmaceutically acceptable” means approved by a regulatory agency of the Federal or a state government or listed in the U.S. Pharmacopeia (2009) of other generally recognized international pharmacopeia for use in animals, and more particularly in humans. Various pharmaceutically acceptable excipients can be used. In some embodiments, the pharmaceutically acceptable excipient can be, but is not limited to, a stiffening agent, a solvent, an emulsifier, a buffering agent, a filler, an emollient, a stabilizer, or combinations thereof.

The term “stiffening agent” refers to a substance, or mixture of substances, added to make the composition more viscous at room temperature. In some embodiments, a stiffening agent is any substance that promotes formation of a formulation having a semi-solid or solid consistency. The stiffening agent can be hydrophilic (e.g., carbopol, carboxymethylcellulose, hydroxypropylmethylcellulose, alginate, polyethylene glycol). In some embodiments, the stiffening agent has low hydrophilic-lipophilic balance (HLB). Examples of suitable stiffening agents include, but are not limited to, hydrogenated vegetable oil, cetyl alcohol, cetyl esters wax, microcrystalline wax, paraffin, stearyl alcohol, lauryl alcohol, myristal alcohol, cetostearyl alcohol, white wax, yellow wax, beeswax, candelilla wax, cotton wax, carnauba wax, bayberry wax, rice-bran wax, and combinations thereof.

The term “solvent” refers to any substance capable of dissolving or dispersing the compound of Formula I or one or more of the excipients. The solvent can be lipophilic. In some embodiments, the solvent is lipophilic, and is 2% to 50% by weight, or 5% to 20% by weight, of the total composition. In some embodiments, the solvent is an oil, such as vegetable, nut, and seed oils (e.g., almond oil, castor oil, coconut oil, corn oil, cotton seed oil, jojoba oil, linseed oil, grape seed oil, rape seed oil, mustard oil, olive oil, palm and palm kernel oil, peanut oil, safflower oil, sesame oil, soybean oil, sunflower-seed oil, crambe oil, wheat germ oil, and cocoa butter), or hydrocarbon and petroleum oils (e.g., petrolatum, mineral oil, and liquid paraffin).

In some embodiments, the composition of the present invention comprises an emulsifier. The term “emulsifier” refers to any substance that promotes formation and stabilization of an emulsion or suspension. In some embodiments, the emulsifier includes, but is not limited to, sodium lauryl sulfate, propylene glycol monostearate, methyl stearate, glyceryl monostearate, and combinations thereof.

The term “buffering agent” refers to any substance capable of neutralizing both acids and bases and thereby maintaining the desired pH of the composition of the present invention. In some embodiments, the buffering agent affects the emulsifying properties. In some embodiments, the buffer can be, but is not limited to, Tris buffers (Tris EDTA (TE), Tris acetate (TAE), Tris phosphate (TPE), Tris glycine), phosphate buffers (e.g., sodium phosphate, potassium phosphate), bicarbonate buffers, acetate buffers (e.g., sodium acetate), ammonium buffers, citrate buffers, and derivatives and combinations thereof. In some embodiments, an organic acid buffer is used. In some embodiments, an acetate buffer, a phosphate buffer, or a citrate buffer can be used. In some embodiments, a zwitterionic buffer can be used. In some embodiments, the buffering agent is a phosphate buffer (e.g., sodium phosphate dibasic).

The pH of the composition of the invention can be physiologically compatible and/or sufficient to maintain stability of the composition. In some embodiments, the composition of the present invention can have a pH of about 5 to about 9, or a pH of about 6.5 to about 8.

As defined herein, a “filler” is a substance used to give bulk to the composition without chemically reacting with the compound of Formula I. Fillers are known to those in the art, see e.g., Remington: The Science and Practice of Pharmacy, 21^{st} ed. (2005).

The concentration of the compound of Formula I in the composition of the present invention can vary. For example, in some embodiments, the compound of Formula I is greater than 40%, 45%, 50%, 60%, 70%, 75%, 80%, 90% or 95% (wt/wt) of the composition. In some embodiments, the compound of Formula I is about 40% to about 60% (wt/wt) of the composition. In some embodiments, the composition comprising about 40% to about 60% (wt/wt), or about 50% (wt/wt) of the compound of Formula I is placed in a capsule, e.g., a gelatin capsule.

As used herein, “administering” refers to placing or delivering a pharmaceutically effective amount of the compound of Formula I to the subject being treated. Examples of such administration include providing the desired active agent by routes, such as, but not limited to, parenterally, subcutaneously, intravenously, intramuscularly, transdermally, buccally, or orally. For example, composition of the present invention can be administered via solid oral dosage forms which include, but are not limited to, tablets, caplets, coated tablets, capsules, cachets, pellets, pills, powders, granules, syrups, slurries, and liquids; topical dosage forms which include, but are not limited to, transdermal patches, powders, fluid emulsions, fluid suspensions, semi-solids, ointments, pastes, creams, gels and jellies, and foams; and parenteral dosage forms which include, but are not limited to, solutions, suspensions, emulsions, and dry powder. The means and methods for administration are known in the art and an artisan can refer to various pharmacologic references for guidance. For example, “Modern Pharmaceutics,” Banker & Rhodes, Marcel Dekker, Inc., 4^{th} ed. (2002); and “Goodman & Gilman's The Pharmaceutical Basis of Therapeutics,” 10th ed., MacMillan Publishing Co., New York 2001 can be consulted.

In some embodiments, the composition is administered orally, e.g., the composition can be administered via an oral dosage form. The dosage form can include, e.g., a push-fit capsule, or a soft sealed capsule. In some embodiments, the capsule is made of gelatin, or gelatin and a plasticizer, such as glycerol or sorbitol. The push-fit capsules can contain the active ingredients in admixture with filler such as, e.g., oils, tocopherol derivatives, lactose, binders such as, e.g., starches, and/or lubricants such as, e.g., talc or magnesium stearate and/or stabilizers. In soft capsules, the compound of Formula I can be dissolved or suspended in suitable liquids, such as fatty oils, liquid paraffin, or liquid polyethylene glycols. In some embodiments, oral administration is accomplished by administering to the subject a liquid dosage form. Liquid dosage forms for oral administration can include pharmaceutically acceptable emulsions, solutions, suspensions, syrups, and elixirs containing inert diluents commonly used in the art, such as water. The composition can also be administered in liposome formulations. In some embodiments, oral administration is accomplished by administering to the subject a solid oral dosage form. Solidifying agents are known in the art, and can include, e.g., polyethylene glycol glycerides composed of mono-, di-, and triglycerides, and mono- and diesters of polyethylene glycol (Gelucire®, Gattefossé Canada, Montreal, Canada) and Neusilin® (magnesium aluminometasilicate; Fuji Chemical Co., Japan). All compositions for oral administration should be in dosages suitable for such administration.

The composition of the present can also be administered transdermally. Transdermal administration of the composition of the present invention can be applied to a plaster or a transdermal patches, both of which are known in the art, for prolonged delivery across the skin. Devices or systems known to the art include reservoir type devices involving membranes that control the rate of drag release to the skin and devices involving a dispersion of the drug in a matrix.

The composition can also be administered in parenteral dosage forms, i.e., via intravenous, intraarterial, intramuscular, or intraperitoneal dosage forms. Parenteral preparations, for example, sterile injectable aqueous or oleaginous suspensions, can be formulated according to the known art using suitable dispersing or wetting agents and suspending agents. The sterile injectable preparation can also be a sterile injectable solution or suspension in a nontoxic parenterally acceptable diluent or solvent, for example, as a solution in propylene glycol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The amount of active ingredient that can be combined with the carrier materials to produce a single dosage form will vary depending upon the host to which the active ingredient is administered and the particular mode of administration. It will be understood, however, that the specific dose level for any particular patient will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The therapeutically effective amount or effective amount for a given situation can be readily determined by routine experimentation and is within the skill and judgment of the ordinary clinician.

In some embodiments, an “effective amount” of a compound of Formula I is an amount of the compound sufficient to modulate, normalize, or enhance one or more energy biomarkers. A “therapeutically effective amount” of a compound of Formula I is an amount of the compound, which, when administered to a subject, is sufficient to reduce or eliminate either a disease or one or more symptoms of a disease, or to retard the progression of a disease or one or more symptoms of a disease, or to reduce the severity of a disease or one or more symptoms of a disease, or to suppress the clinical manifestation of a disease, or to suppress the manifestation of adverse symptoms of a disease. A therapeutically effective amount can be given in one or more administrations. An “effective amount” of a compound embraces both a therapeutically effective amount, as well as an amount effective to modulate, normalize, or enhance one or more energy biomarkers in a subject.

The term “daily dosage,” “daily dosage level,” “daily dosage amount,” or “daily dose” means the total amount of the compound of Formula I (or a stereoisomer thereof) administered per day. Thus, for example, administration of an compound of Formula I to a subject at a “daily dosage amount of 30 g” means that the subject receives a total of 30 g of the compound on a daily basis, whether the compound is administered as a single 30 g dose or, e.g., three separate 10 g doses. Conventional means of administering the compound of Formula I can a single daily oral dose, a twice daily dosing, three times daily dosing, or four times daily dosing. The term “once daily,” or “daily” refers to administration of a composition of the present invention once during a 24 hour period.

Daily dosage amounts of the compound of Formula I can vary, but can include, e.g., about 0.5 μg/kg to about 200 mg/kg body weight, or about 1.0 μg/kg to about 100 mg/kg body weight, or about 2.0 μg/kg to about 50 mg/kg body weight, or about 3.0 μg/kg to about 10 mg/kg body weight, or about 100.0 μg/kg to about 10 mg/kg body weight, or about 1.0 mg/kg to about 10 mg/kg body weight, or about 10 mg/kg to about 100 mg/kg body weight, or about 50 mg/kg to about 150 mg/kg body weight, or about 100 mg/kg to about 200 mg/kg body weight, or about 150 mg/kg body weight.

In some embodiments, various administration regimens can be used to achieve the desired beneficial effects. In some embodiments, the composition of the present invention is administered for treatment of a chronic disease, and thus is administered at least once daily for the remainder of the subject's lifetime, or from 1 to 20 years, or 1, 2, 5, 10, or 15 years. In some embodiments, the composition is used to achieve a more immediate beneficial effect on the subject, and the composition is administered daily for at least 1 week, 2 weeks, 3 weeks, 1 month, 2 months, 6 months, or 9 months to the subject. In some embodiments, administration is “continuous” or “consecutive” for the length of the treatment period. The term “continuous” or “consecutive” in reference to “administration” means that the frequency of administration is at least once daily. Thus, e.g., the phrase “the composition is administered continuously for more than three weeks” indicates that the composition is administered at least once daily for at least 21 consecutive calendar days. Note, however, that the frequency of administration can be greater than once daily and still be “consecutive,” e.g., twice or even three times daily. Additionally, administration of the composition for “consecutive” days can be achieved by dosage forms that administer the composition for longer than a single day. For example, a single transdermal patch that delivers a daily dosage amount of the compound of Formula I for 7 consecutive days would be considered to have “administered the compound for 7 consecutive days.”

The terms “treat” and “treatment” refer to both therapeutic treatment and prophylactic or preventative measures, wherein the object is to prevent, inhibit, reverse or slow down (lessen) an undesired physiological condition, disorder or disease, or obtain beneficial or desired clinical results. For purposes of this invention, beneficial or desired clinical results include, but are not limited to, alleviation of symptoms; diminishment of extent of condition, disorder or disease; stabilized (i.e., not worsening) state of condition, disorder or disease; delay in onset, or slowing, of condition, disorder or disease progression; amelioration of the condition, disorder or disease state, remission (whether partial or total); or enhancement or improvement of condition, disorder or disease. Treatment also includes, but is not limited to, eliciting a cellular response that is clinically significant, without excessive levels of side effects. “Treating” a disease with the compounds and methods discussed herein is defined as administering one or more of the compounds discussed herein, with or without additional therapeutic agents, in order to reduce or eliminate either the disease or one or more symptoms of the disease, or to retard the progression of the disease or of one or more symptoms of the disease, or to reduce the severity of the disease or of one or more symptoms of the disease. “Suppression” of a disease with the compounds and methods discussed herein is defined as administering one or more of the compounds discussed herein, with or without additional therapeutic agents, in order to suppress the clinical manifestation of the disease, or to suppress the manifestation of adverse symptoms of the disease. The distinction between treatment and suppression is that treatment occurs after adverse symptoms of the disease are manifest in a subject, while suppression occurs before adverse symptoms of the disease are manifest in a subject. Suppression can be partial, substantially total, or total. Because many of the mitochondrial disorders are inherited, genetic screening can be used to identify patients at risk of the disease. The compounds and methods of the invention can then be administered to asymptomatic patients at risk of developing the clinical symptoms of the disease, in order to suppress the appearance of any adverse symptoms.

“Subject” refers to human, and nonhuman animals, e.g., domestic and farm animals, and zoo, sports, and companion animals such as household pets and other domesticated animals such as, but not limited to, cattle, sheep, ferrets, swine, horses, rabbits, goats, dogs, cats and the like. In some embodiments, companion animals are dogs and cats.

The compounds can be useful in treating or suppressing mitochondrial disorders, and methods of using such compounds for modulation of energy biomarkers. “Modulation” of, or to “modulate,” an energy biomarker means to change the level of the energy biomarker towards a desired value, or to change the level of the energy biomarker in a desired direction (e.g., increase or decrease). Modulation can include, but is not limited to, normalization and/or enhancement.

“Normalization” of, or to “normalize,” an energy biomarker is defined as changing the level of the energy biomarker from a pathological value towards a normal value, where the normal value of the energy biomarker can be 1) the level of the energy biomarker in a healthy person or subject, or 2) a level of the energy biomarker that alleviates one or more undesirable symptoms in the person or subject. For example, to normalize an energy biomarker in a subject which is depressed in a disease state means to increase the level of the energy biomarker towards the normal (healthy) value or towards a value which alleviates an undesirable symptom.

“Enhancement” of, or to “enhance,” energy biomarkers means to intentionally change the level of one or more energy biomarkers away from either the normal value, or the value before enhancement, in order to achieve a beneficial or desired effect. For example, in a situation where significant energy demands are placed on a subject, it can be desirable to increase the level of ATP in that subject to a level above the normal level of ATP in that subject. Enhancement can also be of beneficial effect in a subject suffering from a disease or pathology such as a mitochondrial disease, in that normalizing an energy biomarker can not achieve the optimum outcome for the subject; in such cases, enhancement of one or more energy biomarkers can be beneficial, for example, higher-than-normal levels of ATP, or lower-than-normal levels of lactic acid (lactate) can be beneficial to such a subject.

### Examples

Alpha-tocopheryl acetate (Vita-Solar Biotechnology Co., China) was hydrolyzed to achieve the non-isolated intermediate of alpha-tocopherol by charging the appropriate quantity of R,R,R-alpha-tocopheryl acetate (7.0 kg) into a 72 L reaction vessel. The alpha-tocopheryl acetate was dissolved in ethanol (200 Proof denatured with 0.05% toluene). The reaction vessel was cooled to 10-12° C. and potassium hydroxide pellets (≧85% A.C.S. grade) were added to the flask while the temperature was maintained at 10 to 15° C. The mixture was agitated for over 1 hour. After at least one hour, the reaction was checked for completeness by sampling the batch and analyzing by HPLC analysis.

The reaction was completed when alpha-tocopheryl acetate was less than 0.5% area percent at 205 nm. The hydrolysis reaction was neutralized with hydrochloric acid solution (34% to 39% aqueous) to give alpha-tocopherol in the reaction vessel. The alpha-tocopherol solution was transferred to a 100 L separatory funnel into which tert-butyl methyl ether (TBME) was added (17.8 kg).

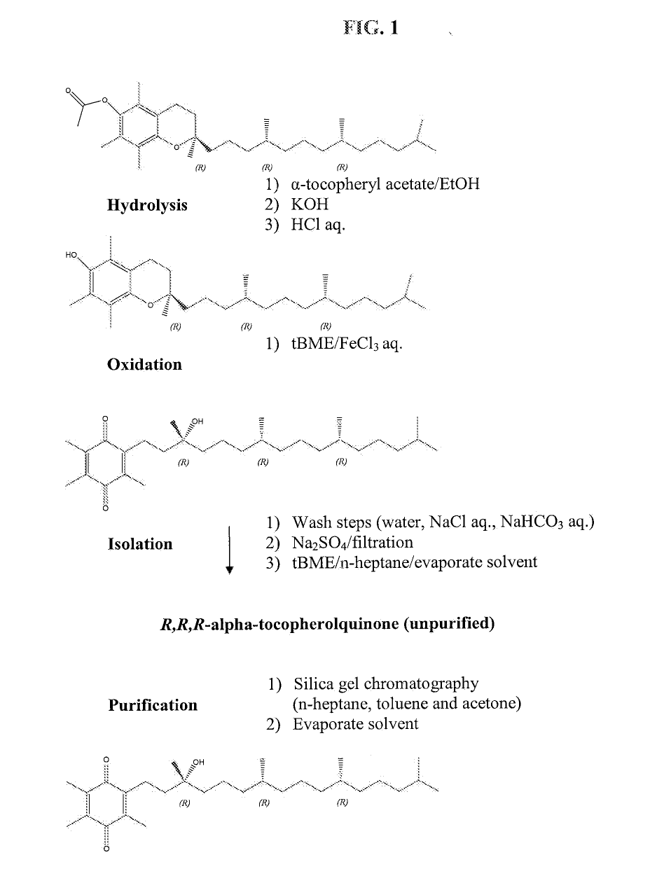

Ferric chloride solution was added in four portions, each portion providing 0.75 equivalents of ferric chloride per unit of alpha-tocopherol, resulting in an overall metal salt oxidizing agent/alpha-tocopherol ratio of 3. The first aliquot was added into the separatory funnel and stirred for approximately 35±5 minutes. The mixture was allowed to settle and the aqueous layer was removed to waste. Ferric chloride additions were carried out three more times for a total of four aliquots. After the last aqueous layer was removed to waste, the TBME containing the resulting R,R,R-alpha-tocopherolquinone was washed with water four times. To carry out the wash, 8.9 kg of water was added to the 100 L separatory funnel and agitated for 10 minutes. The aqueous layer was removed to waste. The batch was sampled and analyzed by an in-process HPLC method. See FIG. 1 for an overview of the process.

HPLC analysis was accomplished using the conditions in Table 1:

TABLE 1

| Column | Gemini C6-Phenyl column | |
|---|---|---|
| | (4.6 mm diameter × 150 mm length, 3 μm particle | |
| | size). | |
| Column | 30° C. | |
| temperature | | |
| Flow | 1.0 mL/minute | |
| Mobile Phase A | 80:20 v/v acetonitrile:water | |
| Mobile Phase B | acetonitrile | |
| Gradient elution | Time | % B |
| | 0 | 0 |
| | 30 | 100 |
| | 50 | 100 |
| | 51 | 0 |

| | |
|---|---|
| Injection volume | 10 μL |
| Detection | UV at 261 nm and 205 nm |

The TBME containing alpha-tocopherolquinone was then washed with USP purified water containing A.C.S. reagent grade sodium chloride (1.5 kg of NaCl in 7.4 kg of water) by adding the sodium chloride solution to the 100 L funnel and agitating for a minimum of 5 minutes. The sodium chloride solution was then removed to waste.

The TBME containing alpha-tocopherolquinone was then washed with water containing sodium bicarbonate (A.C.S. reagent grade) (150 g of sodium bicarbonate to 8.9 kg of water) by adding the sodium bicarbonate to the 100 L separatory funnel and agitating for a minimum of 5 minutes. The sodium bicarbonate solution was then removed to waste.

The organic layer was drained into 5 gallon polyethylene containers and a sufficient quantity of granular sodium sulfate was added to each container so that 0.5 inch to 1 inch was on the bottom of the containers. The organic layer (alpha-tocopherolquinone in TBME) was charged into a rotary evaporator and the solvent was removed under vacuum at 35° C. Once the TBME was evaporated, three kilograms of A.C.S. reagent grade n-heptane was added to the evaporation flask under vacuum, and evaporated for a minimum of 1 hour until no more distillate was seen.

The crude product was dissolved in and rinsed out of the evaporation flask with A.C.S. reagent grade n-heptane. The product in n-heptane was stored in 5-gallon high density polyethylene containers prior to purification.

It is to be appreciated that the Detailed Description section, and not the Summary and Abstract sections, is intended to be used to interpret the claims. The Summary and Abstract sections can set forth one or more but not all exemplary embodiments of the present invention as contemplated by the inventor(s), and thus, are not intended to limit the present invention and the appended claims in any way.

All of the various embodiments or options described herein can be combined in any and all variations. The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

1. A method of synthesizing a compound of Formula I: 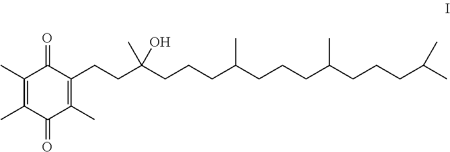
or a stereoisomer thereof, the method comprising oxidizing alpha-tocopherol with a metal salt oxidizing agent to form the compound of Formula I, wherein a stoichiometric ratio (mol/mol) of metal salt oxidizing agent/alpha-tocopherol is 1.6 to 4.

2. A method of synthesizing a compound of Formula I: 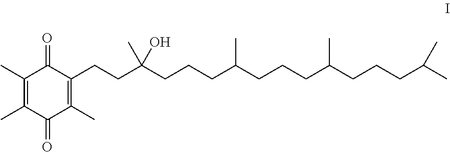
or a stereoisomer thereof, the method comprising:
(a) hydrolyzing alpha-tocopheryl acetate in the presence of a base;
(b) neutralizing the hydrolyzing of (a), thereby forming alpha-tocopherol; and
(c) oxidizing the alpha-tocopherol of (b) with a metal salt oxidizing agent to form the compound of Formula I, wherein a stoichiometric ratio (mol/mol) of metal salt oxidizing agent/alpha-tocopherol is 1.6 to 4.

3. The method of claim 1, wherein the metal salt oxidizing agent is an iron halide.

4. The method of claim 3, wherein the iron halide is FeCl₃.

5. The method of claim 1, wherein the metal salt oxidizing agent is serially added in more than one portion.

6. The method of claim 1, wherein the stoichiometric ratio (mol/mol) of metal salt oxidizing agent/alpha-tocopherol is 2.5 to 3.5.

7. The method of claim 1, wherein the metal salt oxidizing agent is added in an amount sufficient to oxidize 70% (mol/mol) to 98% (mol/mol) of the alpha-tocopherol to the compound of Formula I.

8. The method of claim 2, further comprising washing the product of (c) with an aqueous solution.

9. The method of claim 2, wherein the base is potassium hydroxide or sodium hydroxide.

10. The method of claim 2, wherein the neutralizing comprises addition of an acid.

11. The method of claim 2, wherein the alpha-tocopheryl acetate is dissolved in an alcohol before the hydrolyzing.

12. The method of claim 2, wherein the hydrolyzing occurs at a temperature ranging from about 5° C. to about 20° C.

13. The method of claim 2, wherein the alpha-tocopheryl acetate is R,R,R-alpha-tocopheryl acetate.

14. The method of claim 1, wherein the compound of Formula I is 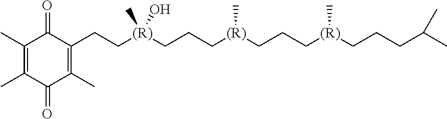
or a stereoisomer thereof.

15. The method of claim 2, wherein the alpha tocopheryl acetate is isolated from a plant.

16. A compound of Formula I, made by the method of claim 1.

17. A composition comprising the compound of claim 16 and an excipient, wherein the composition has less than 2% (mol/mol) gamma-tocopherolquinone relative to the compound of Formula I.

18. The composition of claim 17, wherein the compound of Formula I is: 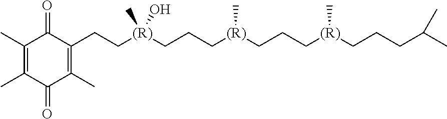
or a stereoisomer thereof.

19. The composition of claim 17, wherein the composition has less than 0.7% (mol/mol) gamma-tocopherolquinone relative to the compound of Formula I.

20. The composition of claim 17, wherein the composition has less than 0.2% (mol/mol) gamma-tocopherolquinone relative to the compound of Formula I.

21. The composition of claim 17, wherein the compound of Formula I is greater than 70% (wt/wt) of the composition.

22. The composition of claim 17, wherein the composition has less than 20% (wt/wt) of alpha-tocopheryl acetate, alpha-tocopherol, beta-tocopherol, beta-tocopherolquinone, gamma-tocopherol, or combinations thereof.

23. The composition of claim 17, wherein the composition is a pharmaceutically acceptable composition.

24. (canceled)

25. An oral dosage form comprising the composition of claim 23.

26. A method of treating a mitochondrial disorder, modulating one or more energy biomarkers, normalizing one or more energy biomarkers, or enhancing one or more energy biomarkers, comprising administering to a subject a therapeutically effective amount or effective amount of the composition of claim 23.

27. The method of claim 26, wherein the compound of Formula I is 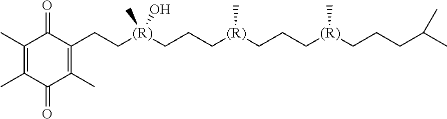
or a stereoisomer thereof.

28. The method of claim 26, comprising treating the mitochondrial disorder, wherein the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Leigh Disease; Kearns-Sayre Syndrome (KSS); Friedreich's Ataxia (FA); other myopathies; cardiomyopathy; encephalomyopathy; renal tubular acidosis; neurodegenerative diseases; Parkinson's disease; Alzheimer's disease; amyotrophic lateral sclerosis (ALS); motor neuron diseases; other neurological diseases; epilepsy; genetic diseases; Huntington's Disease; mood disorders; schizophrenia; bipolar disorder; age-associated diseases; macular degeneration; diabetes; and cancer.

29. The method of claim 26, comprising treating the mitochondrial disorder, wherein the mitochondrial disorder is selected from the group consisting of inherited mitochondrial diseases; Myoclonic Epilepsy with Ragged Red Fibers (MERRF); Mitochondrial Myopathy, Encephalopathy, Lactacidosis, Stroke (MELAS); Leber's Hereditary Optic Neuropathy (LHON); Leigh Disease; Kearns-Sayre Syndrome (KSS); and Friedreich's Ataxia (FA).

30. The method of claim 26, comprising modulating one or more energy biomarkers, normalizing one or more energy biomarkers, or enhancing, one or more enemy biomarkers, wherein the energy biomarker is selected from the group consisting of: lactic acid (lactate) levels, either in whole blood, plasma, cerebrospinal fluid, or cerebral ventricular fluid; pyruvic acid (pyruvate) levels, either in whole blood, plasma, cerebrospinal fluid, or cerebral ventricular fluid; lactate/pyruvate ratios, either in whole blood, plasma, cerebrospinal fluid, or cerebral ventricular fluid; phosphocreatine levels, NADH (NADH+H³⁰) levels; NADPH (NADPH+H³⁰) levels; NAD levels; NADP levels; ATP levels; reduced coenzyme Q (CoQ^{red}) levels; oxidized coenzyme Q (CoQ^{OX}) levels; total coenzyme Q (CoQ^{tot}) levels; oxidized cytochrome C levels; reduced cytochrome C levels; oxidized cytochrome C/reduced cytochrome C ratio; acetoacetate levels, β-hydroxy butyrate levels, acetoacetate/β-hydroxy butyrate ratio, 8-hydroxy-2′-deoxyguanosine (8-OHdG) levels; levels of reactive oxygen species; levels of oxygen consumption (VO₂); levels of carbon dioxide output (VCO₂); respiratory quotient (VCO₂/VO₂); exercise tolerance; and anaerobic threshold.

31. The method of claim 26, wherein the subject is selected from the group consisting of: a subject with a mitochondrial disease; a subject undergoing strenuous or prolonged physical activity; a subject with chronic energy problems; a subject with chronic respiratory problems; a pregnant female; a pregnant female in labor; a neonate; a premature neonate; a subject exposed to an extreme environment; a subject exposed to a hot environment; a subject exposed to a cold environment; a subject exposed to an environment with lower-than-average oxygen content; a subject exposed to an environment with higher-than-average carbon dioxide content; a subject exposed to an environment with higher-than-average level of air pollution; a subject with lung disease; a subject with lower-than-average lung capacity; a tubercular patient; a lung cancer patient; an emphysema patient; a cystic fibrosis patient; a subject recovering from surgery; a subject recovering from illness; a subject undergoing acute trauma; a subject in shock; a subject requiring acute oxygen administration; a subject requiring chronic oxygen administration; an elderly subject; an elderly subject experiencing decreased energy; and a subject suffering from chronic fatigue.
